(19) United States
(12) Patent Application Publication
(10) Pub. No.: US 2002/0107528 A1
(43) Pub. Date: Aug. 8, 2002

(54) Device for cleansing the body

(76) Inventor: Sophie Helene Vayrette, Courbevoie (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/043,342

(22) Filed: Jan. 14, 2002

(30) Foreign Application Priority Data
Jan. 12, 2001 (FR) 01 00409

(51) Int. Cl. A61B 17/50

(52) U.S. Cl. 606/131

(57) ABSTRACT

A device for treating a keratinous surface of a body may comprise an envelope defining an opening configured to receive a users hand. The envelope may comprise a first outer face comprising a nonwoven fibrous material and having a first roughness and a second outer face comprising a nonwoven fibrous material and having a second roughness that differs from the first roughness.

The present invention relates to a device for treating a body. In particular, the invention relates to a device for cleansing and/or exfoliating a keratinous surface of a body.

Cosmetic and/or care products containing exfoliative particles such as, for example, polyethylene powder or fine particles of quartz or of nut shell, or the like, may be used to remove dead skin cells. However, such products typically are sold without an applicator, and so users are obliged to use a traditional washing cloth, a sponge, and/or their hands to apply the product.

Gloves designed to apply cosmetic products exist. For example, U.S. Pat No. 4,347,931 describes a mitten with an impregnated fluffy face. The mitten is made from an impermeable thermoplastic film partially covered with a woven or nonwoven fluffy coating. Such a mitten does not have a face having a roughness sufficient to allow the removal of dead skin cells by exfoliation. Moreover, the impregnation of the whole mitten, either by the user or beforehand during its manufacture, generally must be performed by applying the product to each face of the mitten, given that the mitten is made of impermeable material.

It may be desirable to produce a device for treating the keratinous surface of a body that does not have the drawbacks of the prior art, and that may be made relatively cheaply while being practical to use.

It also may be desirable to provide a device for treating the body, and in particular exfoliating the body, that can be used independently, for example, without the need to add additional product.

It may further be desirable to provide a device for treating a keratinous surface of the body that may be relatively easily impregnated with a cosmetic and/or care product throughout substantially its whole structure.

It should be understood that the invention could be practiced without performing one or more of the aspects described above. Other aspects will become apparent from the detailed description which follows. As embodied and broadly described herein, a device for treating a keratinous surface of a body may comprise an envelope defining an opening configured to receive a users hand. The envelope may comprise a first outer face comprising a nonwoven fibrous material and having a first roughness and a second outer face comprising a nonwoven fibrous material and having a second roughness that differs from the first roughness.

The nonwoven fibrous materials referred to herein include materials made of a plurality of fibers, wherein the materials are formed other than by weaving the fibers together. The materials may be formed by a variety of techniques known to those skilled in the art for forming webs of nonwoven fibrous material, including, for example, conventional papermaking techniques, air-laid processes, and/or a combination of such techniques followed by heat treatment to enhance the roughness of the material. Of course, these techniques are exemplary only and nonwoven fibrous materials formed by other techniques are considered to be within the scope of the invention.

The term “face” is used broadly and may include any outer surface portion of the envelope, including outer surface portions that may face in the same direction or in opposite directions, such as the respective opposite faces corresponding to the palm of a user's hand and the back of the user's hand when the envelope is in the form of a glove or mitten, for example. As an alternative, for an envelope that may have more than two planar sides, such as a cube-shaped or other similarly shaped envelope, for example, the outer faces may include surface portions that face in the same direction or that are adjacent to one another and face in differing directions, including directions other than opposite directions. As an example, the outer faces of a cube-shaped envelope may include surface portions that face in directions that are ninety degrees apart from each other.

To choose two sheets of differing roughnesses (for example, two sheets whose surface state is more or less bumpy), a method may be used for measuring the surface state so as to compare the roughness of different sheets. It may be possible, for example, to measure the surface profile using a diamond tip configured to slide over the surface to be measured and to follow the various changes in the surface level. The variations obtained may define a curve which may be processed mathematically by a computer to calculate, for example, the mean arithmetic deviation of the profile. A first surface whose mean arithmetic deviation is higher than that of a second surface may have a greater roughness than the second. It is envisioned that the roughnesses may be compared and/or measured by any other suitable roughness measuring devices or techniques that may be known to those skilled in the art, including subjective techniques involving one or more individuals determining the tactile sensation of material(s) on their own skin. It should be understood that the roughnesses and/or materials could be selected without any comparison, measurement, analysis, etc.

The first roughness may be configured so as to exfoliate the keratinous surface of the body. The second roughness may be less than the first roughness and the second outer face may be configured to soothe and/or apply a product to the keratinous surface of the body.

The device may be permeable, for example the whole device may comprise a permeable structure. As used herein, the expression “permeable” structure includes a structure such that when a particular product, such as a cosmetic and/or care product, for example, is placed on the structure, the product passes through it at least partially. For example, when at least the first sheet is permeable, a product placed on the first sheet may impregnate the first sheet, pass the first sheet, and impregnate the second sheet. As an example, the device may be water-permeable.

Depending on the configuration of the device, when it is desired to impregnate the device with a product, it may be possible to apply the product to only one sheet of the device since the product may be able to reach the whole device due to its permeability. This may make it possible to obtain an impregnated device relatively simply and inexpensively when compared with a device having a relatively impermeable structure. In addition, should the user wish to add a cosmetic and/or care product, whether it be a product with an exfoliative, moisturizing, soothing or cleansing function, the user may be able to apply it to either outer surface, since the product may be able to spread over the entire device. On the other hand, if the user wishes to have product on only one outer surface of the device, it may suffice for the user to apply the product when one of the user's hands, or another relatively impermeable separation member, is inside the device envelope.

Although both faces may be permeable, one or both faces may be impermeable.

In an exemplary embodiment, the envelope may be substantially symmetrical about a longitudinal axis of the envelope. For example, the envelope may be configured as a mitten having two independent thumb portions at opposite lateral ends. Such a configuration may prevent or hinder the envelope from rotating on the user's hand when a user applies it to his body. As an alternative, the envelope may be configured as a glove.

The envelope also may be configured as a mitten. Further, the envelope may have a shape chosen from substantially square, substantially rectangular, substantially elliptical, and substantially circular. The envelope may have numerous other shapes as well, the specific shapes described above being exemplary only.

The nonwoven material of each of the first and second outer faces may be chosen from a nonwoven material comprising thermoplastic fibers, for example. The thermoplastic fibers may comprise polyolefin fibers, such as polyolefin fibers chosen from polypropylene fibers, polyethylene fibers, and polyester fibers, for example. As an option, the thermoplastic fibers may be combined with natural fibers, which may comprise cellulose type fibers, for example.

The first outer face may comprise a first nonwoven material and the second outer face may comprise a second nonwoven material that differs from the first nonwoven material.

In an exemplary embodiment, the first outer face may be made of a sheet of nonwoven material having a thickness ranging from about 400 micrometers to about 1000 micrometers, and/or from about 450 micrometers to about 600 micrometers.

In another exemplary embodiment, the second outer face may be made of a sheet of nonwoven material having a thickness ranging from about 300 micrometers to about 1000 micrometers, and/or from about 450 micrometers to about 600 micrometers.

The nonwoven material may therefore make it possible to use relatively thin sheets and to form an envelope from a relatively small amount of material. As such, the device may be relatively inexpensive to make. Such devices may, for example, be intended to be disposable. Furthermore, the devices thus formed may be relatively nonbulky and may be relatively easily inserted into an outer packaging intended, for example, to be placed in a container containing a product.

The first outer face may be made of a first sheet of nonwoven material and the second outer face may be made of a second sheet of nonwoven material. For example, the first sheet and the second sheet may be attached together along their respective peripheral regions. The first sheet and the second sheet may be attached via heat sealing, for example. Such an attachment may be relatively easy to produce and be relatively robust. In an exemplary embodiment, the first sheet of nonwoven material may define the opening and form the first outer face, and the second sheet may be attached to the first sheet to form the second outer face.

At least one of the first outer face and the second outer face may be configured to be impregnated with at least one product, and may be impregnated with at least one product, such as a cosmetic and/or a care product, for example.

The first outer face may be configured to be impregnated with at least one first product and the second outer face may be configured to be impregnated with at least one second product differing from the first product. In this case, i.e., when each of the two sheets is impregnated with a different product, an impermeable member, such as a sheet of impermeable material or a user's hand, for example, may be introduced inside the envelope before impregnating each sheet. This may prevent the two products from mixing.

In an exemplary embodiment, the first outer face is impregnated with at least one first product and the second outer face is impregnated with at least one second product differing from the first product, the first product and second product being chosen from a cosmetic product and/or a care product, for example. The first product and the second product may chosen from an exfoliative product, a moisturizing product, a soothing product, and a cleansing product, for example.

The keratinous surface may comprise skin.

According to another aspect, a method of treating a keratinous surface of a body comprises providing any of the devices described above, massaging a portion of the body to be treated with the first outer face, and massaging a portion of the body with the second outer face.

The term “providing” is used broadly, and refers to, but is not limited to, making available for use, giving, supplying, obtaining, getting a hold of, acquiring, purchasing, selling, distributing, possessing, making ready for use, and/or placing in a position ready for use.

The first roughness may be configured to exfoliate the keratinous surface of the body and the massaging of the portion of the body with the first outer face may occur prior to the massaging of the portion of the body with the second outer face. For instance, it may be possible both to remove dead skin cells by applying the rougher face to an area of the body, and to cleanse or wipe the area by then applying the less rough face. Such a device may be used without exfoliative cosmetic product, since the rougher face may have a roughness that achieves an exfoliative function. It may be possible at least partially to remove the dead cells from the surface of the skin by moving the rougher face over the skin.

The second roughness may be configured to soothe and/or apply a product to the keratinous surface of the body and the massaging of the portion of the body with the second outer face may comprise at least one of soothing and applying a product to the keratinous surface of the body.

According to an exemplary embodiment, the method may further comprise inserting a hand into the opening of the envelope. Further, the method may comprise changing the position of the envelope with respect to the hand so as to change the relative positions of the first outer face and the second outer face with respect to the hand between the massaging with the first outer face and the massaging with the second outer face.

The method also may comprise impregnating at least one of the first outer face and the second outer face with at least one product. The impregnating may comprise impregnating the first outer face with a first product and impregnating the second outer face with a second product that differs from the first product. The method may further comprise inserting a hand into the opening of the envelope and the impregnating of at least one of the first outer face and second outer face may occur while the hand is inserted in the envelope.

As an option, the impregnating may comprise impregnating both the first outer face and the second outer face with the product. For example, the impregnating of both the first outer face and the second outer face may comprise applying the at least one product to one of the first outer face and the second outer face. In an exemplary embodiment, the impregnating of at least one of the first outer face and the second outer face with the at least one product may be performed by other than a user of the device.

The massaging of the portion of the body with the first outer face may comprise exfoliating the portion of the body.

The portion of the body massaged with the first outer face may be one of the same as and differing from the portion of the body massaged with the second outer face.

Yet another aspect includes a system for treating a keratinous surface of a body comprising any of the devices described above and at least one product for treating the keratinous surface. The at least one product may be a cosmetic product and/or a care product, such as an exfoliative product, a moisturizing product, a soothing product, and a cleansing product, for example. The at least one product may comprise a first product differing from a second product.

Aside from the structural and procedural arrangements set forth above, the invention could include a number of other arrangements, such as those explained hereinafter. It is to be understood that both the foregoing description and the following description are exemplary. The accompanying drawings are included to provide a further understanding of the invention and are incorporated in and constitute a part of this specification. The drawings illustrate exemplary embodiments of the invention and, together with the description, serve to explain certain principles. In the drawings,

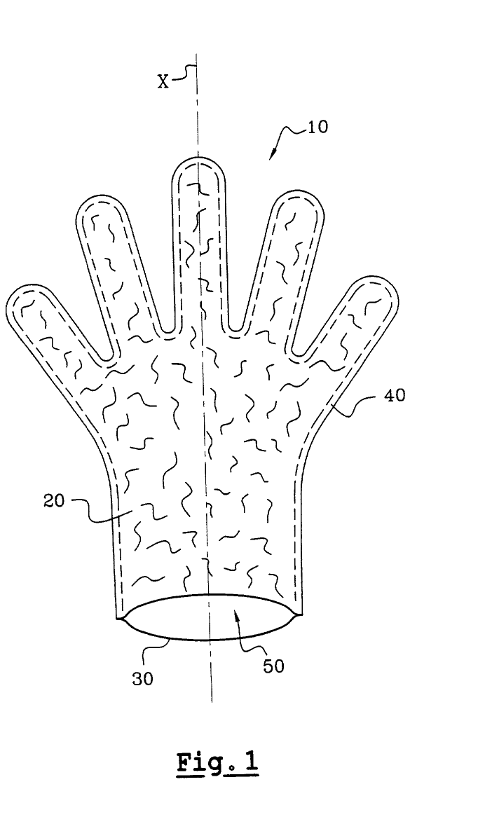

FIG. 1 is a perspective view of an exemplary embodiment of an envelope for treating the body;

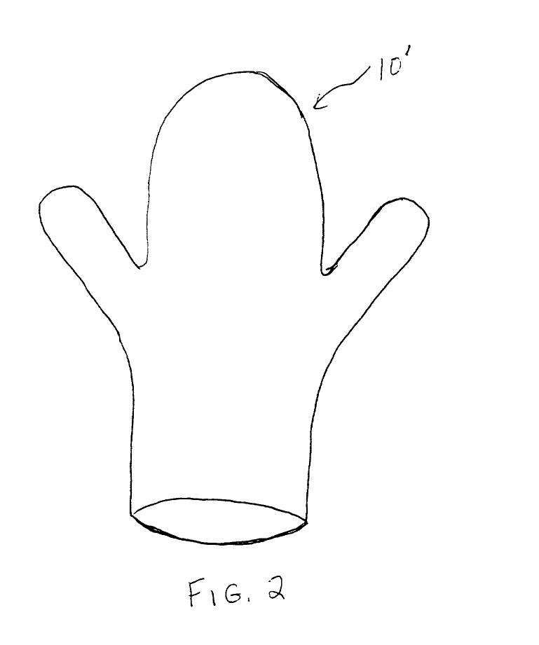

FIG. 2 is a perspective view of another exemplary embodiment of an envelope for treating the body;

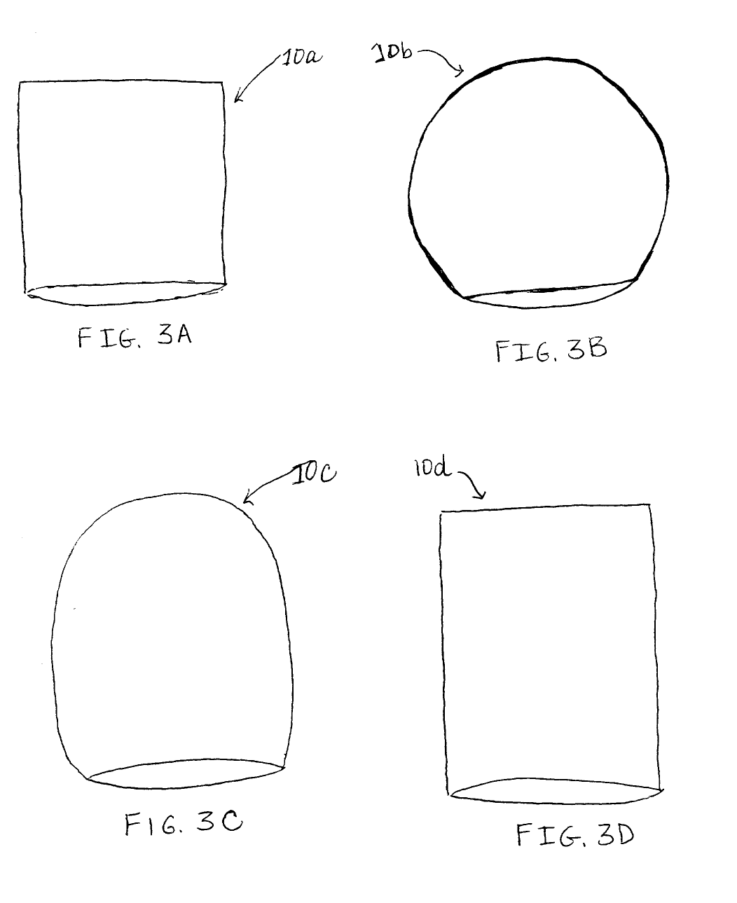

FIGS. 3A-3D are perspective views of other exemplary embodiments of an envelope for treating the body; and

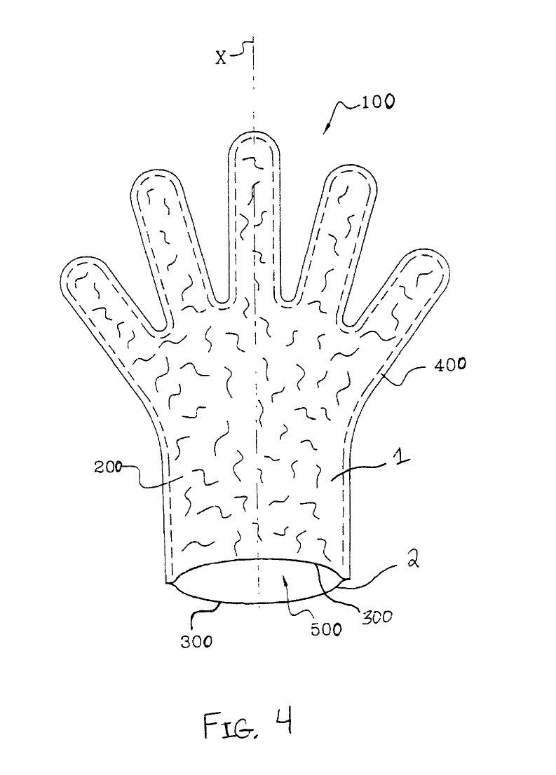

FIG. 4 is a perspective view of yet another exemplary embodiment of an envelope for treating the body.

Reference will now be made in detail to exemplary embodiments, examples of which are illustrated in the accompanying drawings. Wherever possible, the same reference numbers are used in the drawings and the description to refer to the same or like parts.

FIG. 1 shows an envelope 10 in the shape of a five-fingered glove that may be substantially symmetrical about its longitudinal x-axis. The envelope may also be in the shape of an envelope of substantially square, substantially circular, substantially elliptical, or substantially rectangular shape, such as envelopes 10a-10d shown in FIGS. 3A-3D, respectively. Further, the envelope may be in the shape of a mitten with two thumb portions at opposite lateral ends, such as envelope 10′ shown in FIG. 2. Of course, the envelope may have numerous other shapes, including assymetrical shapes such as a mitten having a single thumb portion (not shown). Practically any shape that is suitable for a user to insert his hand to use the envelope for cleansing is envisioned and is within the scope of the invention.

The substantial symmetry of the envelope 10, shown in FIG. 1, may permit the envelope 10 to be used on either the left hand or the right hand. It also may make it possible to obtain reversibility of the envelope 10, such that the user can relatively easily rotate the envelope 10 with respect to his hand. In this manner, the user may associate the first outer face 1 and then the second outer face 2 of the glove, and/or vice versa, with the palm side of his hand in order to apply the outer faces 1, 2 successively to his body.

In the exemplary embodiment shown in FIG. 1, the envelope 10 may be made from two sheets 20, 30 of nonwoven material. The first sheet 20 may have an abrasive function configured for exfoliation of a keratinous surface. The first sheet 20 may comprise a nonwoven material of fibers of thermoplastic synthetic nature, for example. The fibers may be fibers of polyester, nylon, or polyolefin, for example. They may optionally be combined with natural fibers of a cellulose type. An abrasive layer of polypropylene thermoplastic filaments may be deposited on the fibers. The surface of the first sheet 20 that constitutes the first outer face 1 of the glove may also have embossing. Alternatively, a nonwoven material covered with drops of ink or with bumps of any other type making it possible to obtain a sufficient roughness to cause exfoliation may be used. The abrasive first sheet 20 may have a thickness of about 450 μm. A sheet of nonwoven sold by the company Ahlstrom Dexter & Paper under the reference Hydraspun® 8545 may be used, for example, for the first sheet 20.

The second sheet 30 forming a second outer surface 2 may have a cleansing function allowing dead cells to be removed after exfoliation. The second sheet 30 also may comprise a nonwoven material of fibers of thermoplastic synthetic nature. The fibers may be, for example, polyolefin, nylon, or polyester fibers. Such a sheet 30 may have a relatively soft surface state. The sheet 30 may have a thickness of approximately 560 μm. A sheet of nonwoven sold by the company Ahlstrom Dexter & Paper under the reference Hydraspun® 8552 may be used, for example, for the second sheet 30.

The two sheets 20 and 30 may be heat-sealed along a line 40 corresponding substantially to their respective peripheral regions. In this manner an opening 50 of the envelope is defined by the sheets 20, 30. Further, each sheet 20, 30 forms the respective first outer face 1 and second outer face 2 of the envelope 10. If the two sheets have the same base, heat-welding may occur relatively easily. However, it is clear that joining (i.e., attaching) the sheets 20, 30 may occur via any other means such as, for example, by adhesive bonding, sewing, and/or other suitable joining means.

During use, the user may use the glove 10 without adding product. For example, the user may first apply the first outer face 1 formed from the roughness first sheet 20 to the user's body to remove dead cells. Then, the user may rotate the glove 10 on his hand and use the second outer face 2 formed from the softer sheet 30 to wipe the exfoliated surface. If the user wishes to add an exfoliative product, it can be placed on either of the outer faces 1 and 2. Also, after having applied the first outer face 1 formed from the rougher first sheet 20, the user may place a cleansing, moisturizing, and/or soothing product, for example, onto the second outer face 2. Thus, by means of a single device, the user may accomplish the exfoliation and then wipe the exfoliated surface so as to remove the dead cells, while at the same time applying moisturizer.

Of course, in an alternative exemplary manner, the second outer face 2 may first be used to massage the body, followed by the first outer face 1. Different portions of the body also may be treated, for example, by using one of the outer faces on one portion of the body and the other of the outer faces on the other portion. Using the device in this manner may be especially suitable if one portion of the body requires cleansing and another portion requires exfoliation.

According to an exemplary embodiment, before being packaged, the envelope 10 may be impregnated with a product, such as a cosmetic and/or care product, for example. An exfoliative product, for example, may be introduced into the first rougher sheet 20. A moisturizing product may also be added to the softer second sheet 30.

The envelope 10 may then be packaged in a flexible sachet, cardboard, or plastic sleeve type packaging. Alternatively, the packaging may be in the form of a sleeve of label type which may be positioned on a container of cosmetic product. If the envelope is impregnated with product prior to packaging, a packaging that is impermeable to the product contained in the glove, such as, for example, plastic packaging, may be used.

FIG. 4 shows yet another exemplary embodiment of an envelope 100 in the form of a glove. This embodiment differs from the embodiment described with reference to FIG. 1 in that the opening 500 of the envelope 100 is defined by the second sheet of material 300. In other words, the second sheet 300 has a glove-shaped configuration. The second sheet 300 may form the second outer face 2 and the first sheet of material 200 of greater roughness may be attached to the second sheet 300 on an opposite outer surface of the sheet 300 so as to form the first outer face 1 of the envelope. The first sheet of material 200 and/or the second sheet of material 300 may be nonwoven. The first sheet 200 and the second sheet 300 may be attached by heat sealing, adhesive, sewing, or any other suitable attachment mechanisms. Moreover, the sheets may be attached together either along their peripheral regions and/or other portions along their surfaces. As an alternative (not shown), the opening 500 may be defined by the first sheet 200 of greater roughness with the second sheet 300 being attached so as to form the second outer surface 2 of the envelope 100. Other aspects described above with reference to the envelope 10 of FIG. 1 may be incorporated into the envelope 100, including, for instance, the use of the envelope 100. Moreover, rather than having the glove configuration shown, the envelope 100 may have the various configurations described with reference to FIGS. 2 and 3A-3D.

The envelope discussed with reference to the exemplary embodiments may be used to treat a keratinous surface of a body, optionally using cosmetic and/or care products or not using such products (e.g., massaging or exfoliating without usage of a product). However, it should be understood that use of the envelopes in this manner is exemplary only. For example, the envelope may be used to apply, or remove as the case may be, other products. Also, surfaces other than a keratinous surface of the body may be treated. Further, the envelope may be used in the absence of any product.

The sizes, shapes, and assembly of various structural parts and the materials used to make those parts are illustrative and exemplary only and one having ordinary skill in the art would recognize that these materials and sizes can be changed, for example, to produce different effects or desired characteristics of the envelope. It will be apparent to those skilled in the art that various modifications and variations may be made to the structure and methodology. Thus, it should be understood that the invention is not limited to the exemplary embodiments discussed in the specification. Rather, the present invention is intended to cover modifications and variations.

What is claimed is:

1. A device for treating a keratinous surface of a body, the device comprising: an envelope defining an opening configured to receive a user's hand, the envelope comprising:
a first outer face comprising a nonwoven fibrous material and having a first roughness, and
a second outer face comprising a nonwoven fibrous material and having a second roughness that differs from the first roughness.

2. The device of claim 1, wherein the first roughness is configured so as to exfoliate the keratinous surface of the body.

3. The device of claim 1, wherein the second roughness is less than the first roughness.

4. The device of claim 3, wherein the second outer face is configured to at least one of soothe and apply a product to the keratinous surface of the body.

5. The device of claim 1, wherein the device is permeable.

6. The device of claim 1, wherein the envelope is substantially symmetrical about a longitudinal axis of the envelope.

7. The device of claim 6, wherein the envelope is configured as a mitten having two independent thumb portions at opposite lateral ends.

8. The device of claim 6, wherein the envelope is configured as a glove.

9. The device of claim 1, wherein the envelope is configured as a mitten having two independent thumb portions.

10. The device of claim 1, wherein the envelope is configured as a glove.

11. The device of claim 1, wherein the envelope is configured as a mitten.

12. The device of claim 1, wherein the envelope has a shape chosen from substantially square, substantially rectangular, substantially elliptical, and substantially circular.

13. The device of claim 1, wherein the nonwoven material of each of the first and second outer faces is chosen from a nonwoven material comprising thermoplastic fibers.

14. The device of claim 13, wherein the thermoplastic fibers comprise polyolefin fibers.

15. The device of claim 14, wherein the polyolefin fibers are chosen from polypropylene fibers, polyethylene fibers, and polyester fibers.

16. The device of claim 13, wherein the thermoplastic fibers are combined with natural fibers.

17. The device of claim 16, wherein the natural fibers comprise cellulose type fibers.

18. The device of claim 1, wherein the first outer face comprises a first nonwoven material and the second outer face comprises a second nonwoven material that differs from the first nonwoven material.

19. The device of claim 1, wherein the first outer face is made of a sheet of nonwoven material having a thickness ranging from about 400 micrometers to about 1000 micrometers.

20. The device of claim 1, wherein the first outer face is made of a sheet of nonwoven material having a thickness ranging from about 450 micrometers to about 600 micrometers.

21. The device of claim 1, wherein the second outer face is made of a sheet of nonwoven material having a thickness ranging from about 300 micrometers to about 1000 micrometers.

22. The device of claim 1, wherein the second outer face is made of a sheet of nonwoven material having a thickness ranging from about 450 micrometers to about 600 micrometers.

23. The device of claim 1, wherein the first outer face is made of a first sheet of nonwoven material and the second outer face is made of a second sheet of nonwoven material.

24. The device of claim 23, wherein the first sheet and the second sheet are attached together along their respective peripheral regions.

25. The device of claim 24, wherein the first sheet and the second sheet are attached via heat sealing.

26. The device of claim 23, wherein the first sheet of nonwoven material defines the opening and forms the first outer face, and the second sheet is attached to the first sheet to form the second outer face.

27. The device of claim 1, wherein at least one of the first outer face and the second outer face is configured to be impregnated with at least one product.

28. The device of claim 1, wherein at least one of the first outer face and second outer face is impregnated with at least one product.

29. The device of claim 28, wherein the product is chosen from a cosmetic product and a care product.

30. The device of claim 1, wherein the first outer face is configured to be impregnated with at least one first product and the second outer face is configured to be impregnated with at least one second product differing from the first product.

31. The device of claim 1, wherein the first outer face is impregnated with at least one first product and the second outer face is impregnated with at least one second product differing from the first product.

32. The device of claim 31, wherein the first product and second product are chosen from a cosmetic product and a care product.

33. The device of claim 32, wherein the first product and the second product are chosen from an exfoliative product, a moisturizing product, a soothing product, and a cleansing product.

34. The device of claim 1, wherein the keratinous surface comprises skin.

35. A method of treating a keratinous surface of a body, the method comprising:
providing the device of claim 1;
massaging a portion of the body to be treated with the first outer face; and
massaging a portion of the body with the second outer face.

36. The method of claim 35, wherein the first roughness is configured to exfoliate the keratinous surface of the body and the massaging of the portion of the body with the first outer face occurs prior to the massaging of the portion of the body with the second outer face.

37. The method of claim 36, wherein the second roughness is configured to at least one of soothe and apply a product to the keratinous surface of the body and the massaging of the portion of the body with the second outer face comprises at least one of soothing and applying a product to the keratinous surface of the body.

38. The method of claim 35, further comprising inserting a hand into the opening of the envelope.

39. The method of claim 38, further comprising changing the position of the envelope with respect to the hand so as to change the relative positions of the first outer face and the second outer face with respect to the hand between the massaging with the first outer face and the massaging with the second outer face.

40. The method of claim 35, further comprising impregnating at least one of the first outer face and the second outer face with at least one product.

41. The method of claim 40, wherein the at least one product is chosen from a cosmetic product and a treatment product.

42. The method of claim 41, wherein the at least one product is chosen from an exfoliative product, a moisturizing product, a soothing product, and a cleansing product.

43. The method of claim 40, wherein the impregnating comprises impregnating the first outer face with a first product and impregnating the second outer face with a second product that differs from the first product.

44. The method of claim 40, further comprising inserting a hand into the opening of the envelope, wherein the impregnating of at least one of the first outer face and second outer face occurs while the hand is inserted in the envelope.

45. The method of claim 40, wherein the impregnating comprises impregnating both the first outer face and the second outer face with the product.

46. The method of claim 45, wherein the impregnating of both the first outer face and the second outer face comprises applying the at least one product to one of the first outer face and the second outer face.

47. The method of claim 40, wherein the impregnating of at least one of the first outer face and the second outer face with the at least one product is performed by other than a user of the device.

48. The method of claim 35, wherein the massaging of the portion of the body with the first outer face comprises exfoliating the portion of the body.

49. The method of claim 35, wherein the portion of the body massaged with the first outer face is one of the same as and differing from the portion of the body massaged with the second outer face.

50. A system for treating a keratinous surface of a body, the system comprising:
the device of claim 1; and
at least one product for treating the keratinous surface.

51. The system of claim 50, wherein the at least one product is chosen from a cosmetic product and a care product.

52. The system of claim 51, wherein the at least one product is chosen from an exfoliative product, a moisturizing product, a soothing product, and a cleansing product.

53. The system of claim 50, wherein the at least one product comprises a first product and a second product differing from the first product.

54. The system of claim 53, wherein the first outer face is configured to be impregnated with the first product and the second outer face is configured to be impregnated with the second product.

55. The system of claim 50, wherein the first outer face and the second outer face are configured to be impregnated with the at least one product.

56. The system of claim 55, wherein the at least one product is configured to be applied to the first outer face and the second outer face so as to impregnate both the first outer face and the second outer face.

57. The system of claim 50, wherein at least one of the first outer face and the second outer face is impregnated with the at least one product.

58. The system of claim 57, wherein at least one of the first outer face and the second outer face is impregnated with the at least one product by other than a user of the device.

59. The system of claim 50, wherein the envelope is substantially symmetrical about a longitudinal axis of the envelope.

60. A device for treating a keratinous surface of a body, the device comprising:
an envelope defining an opening configured to receive a user's hand, the envelope comprising
a first outer face having a first roughness, and
a second outer face having a second roughness that differs from the first roughness,
wherein at least one of the first outer face and the second outer face comprises a nonwoven fibrous material.

61. The device of claim 60, wherein the first roughness is configured so as to exfoliate the keratinous surface of the body.

62. The device of claim 60, wherein the second roughness is less than the first roughness.

63. The device of claim 62, wherein the second outer face is configured to at least one of soothe and apply a product to the keratinous surface of the body.

64. The device of claim 60, wherein the device is permeable.

65. The device of claim 60, wherein the envelope is substantially symmetrical about a longitudinal axis of the envelope.

66. The device of claim 65, wherein the envelope is configured as a mitten having two independent thumb portions at opposite lateral ends.

67. The device of claim 65, wherein the envelope is configured as a glove.

68. The device of claim 60, wherein the envelope is configured as a mitten.

69. The device of claim 60, wherein the envelope has a shape chosen from substantially square, substantially rectangular, substantially elliptical, and substantially circular.

70. The device of claim 60, wherein the nonwoven material is chosen from a nonwoven material comprising thermoplastic fibers.

71. The device of claim 70, wherein the thermoplastic fibers comprise polyolefin fibers.

72. The device of claim 71, wherein the polyolefin fibers are chosen from polypropylene fibers, polyethylene fibers, and polyester fibers.

73. The device of claim 70, wherein the thermoplastic fibers are combined with natural fibers.

74. The device of claim 73, wherein the natural fibers comprise cellulose type fibers.

75. The device of claim 60, wherein the first outer face comprises a first nonwoven fibrous material and the second outer face comprises a second nonwoven fibrous material that differs from the first nonwoven fibrous material.

76. The device of claim 60, wherein the first outer face is made of a sheet of nonwoven material having a thickness ranging from about 400 micrometers to about 1000 micrometers.

77. The device of claim 60, wherein the second outer face is made of a sheet of nonwoven material having a thickness ranging from about 300 micrometers to about 1000 micrometers.

78. The device of claim 60, wherein the first outer face is made of a first sheet of material and the second outer face is made of a second sheet of material.

79. The device of claim 78, wherein the first sheet and the second sheet are attached together along their respective peripheral regions.

80. The device of claim 79, wherein the first sheet and the second sheet are attached via heat sealing.

81. The device of claim 78, wherein the first sheet defines the opening and forms the first outer face, and the second sheet is attached to the first sheet to form the second outer face.

82. The device of claim 60, wherein at least one of the first outer face and the second outer face is configured to be impregnated with at least one product.

83. The device of claim 60, wherein at least one of the first outer face and second outer face is impregnated with at least one product.

84. The device of claim 83, wherein the product is chosen from a cosmetic product and a care product.

85. The device of claim 60, wherein the first outer face is configured to be impregnated with at least one first product and the second outer face is configured to be impregnated with at least one second product differing from the first product.

86. The device of claim 60, wherein the first outer face is impregnated with at least one first product and the second outer face is impregnated with at least one second product differing from the first product.

87. The device of claim 86, wherein the first product and second product are chosen from a cosmetic product and a care product.

88. The device of claim 87, wherein the first product and the second product are chosen from an exfoliative product, a moisturizing product, a soothing product, and a cleansing product.

89. The device of claim 60, wherein the keratinous surface comprises skin.
